(19) United States
(12) Patent Application Publication
(10) Pub. No.: US 2019/0287675 A1
(43) Pub. Date: Sep. 19, 2019

(54) SYSTEMS AND METHODS FOR DETERMINING HEALTHCARE QUALITY MEASURES BY EVALUTATING SUBJECT HEALTHCARE DATA IN REAL-TIME

(71) Applicant: KONINKLIJKE PHILIPS N.V., EINDHOVEN (NL)

(72) Inventors: Erina GHOSH, Boston, MA (US); Oladimeji Feyisetan FARRI, Yorktown Heights, NY (US); Lin YANG, Chandler, AZ (US); Eric Thomas CARLSON, New York, NY (US)

(21) Appl. No.: 16/301,618

(22) PCT Filed: May 30, 2017
(86) PCT No.: PCT/EP2017/062988

§ 371 Date: Nov. 14, 2018

Related U.S. Application Data
(60) Provisional application No. 62/348,160, filed on Jun. 10, 2016.

(51) Int. Cl. G16H 40/20; G16H 10/20; G06F 16/2455

(52) U.S. Cl. CPC G16H 40/20; G06F 16/24564; G16H 10/20

(57) ABSTRACT

The present disclosure pertains to obtaining information that facilitates determining healthcare quality measures by evaluating subject healthcare data in real-time. Information is obtained that facilitates determination of compliance with healthcare quality measures. This is accomplished by running queries on a clinical database comprising subject healthcare data. Natural language processing is utilized to extract subject healthcare data at various times from the clinical database based on individual queries, thus determining any changes in subject healthcare data over time. A rule-based component is used to implement healthcare quality measures and evaluate updated subject healthcare data based upon rules.

### BACKGROUND

### 1. Field

The present disclosure relates to systems and methods for determining quality measures related to healthcare by evaluating subject healthcare data in real-time.

### 2. Description of the Related Art

It is well known that compliance with clinical quality measures is an important metric in determining hospital reimbursement. Typically, compliance statistics are determined retrospectively and the compliance information is unavailable in real-time. Therefore, there is an increased risk in well-informed decision making in real-time.

The Centre for Medicare and Medicaid Services (CMS) implemented quality initiatives to assure quality healthcare. CMS and other organizations defined quality measures to implement different quality initiatives. CMS uses quality measures to quantify healthcare quality improvement, and assess pay for reporting and for public reporting of hospital performance. Quality measures are tools that help people to measure or quantify healthcare processes, outcomes, subject perceptions, and organizational structure and/or systems that are associated with the ability to provide high-quality healthcare and/or that relate to one or more quality goals for healthcare. These goals may include such things as effective, safe, efficient, subject-centered, equitable, and timely care.

Hospitals get reimbursed for compliance with these measures. The burden of reporting compliance statistics is on the hospitals. Typically, hospitals extract data on measure compliance from electronic health records (EHRs) a few times in a year. As a result, calculations are performed retrospectively and there is a lag between when the care was provided and when statistics were determined. While the data needed to determine measure compliance is in the EHRs, it is very difficult to extract the data and perform the calculations automatically. Currently, data extraction and measure compliance calculations are performed manually by independent evaluators.

Evaluating compliance to quality measures requires collecting multiple data types including such things as labs, medications, information on current diagnoses, and past medical history. This information is available in the subject EHR, but accessing this information is challenging. An important step is determining which quality measures are applicable to which subjects. This involves interpreting numeric measurements in the context of structured and unstructured text. Determining which measures are applicable requires expert knowledge. Ergo, this is typically done manually and it is a time-consuming and expensive process.

Given the difficulty in calculating compliance statistics, they have usually been calculated once (or a few times) per year for reporting purposes. Since compliance statistics are not calculated in real-time, nurses and physicians do not know whether subject care is in compliance or not during a subject's stay. Currently available tools typically determine compliance statistics in a retrospective manner and group together information from multiple subjects in a unit or hospital to present an overview. However, information on individual subject care is typically unavailable in real-time, which hinders effective, guideline-based decision-making Having access to this information would facilitate better-informed care services for the target subject rather than future actions based on quality measures. This would result in better quality of care and improved compliance with CMS quality measures.

### SUMMARY

Accordingly, it is an object of one or more embodiments of the present invention to provide a system configured for determining healthcare quality measures by evaluating subject healthcare data in real-time. The system comprises one or more hardware processors configured by machine-readable instructions to obtain information that facilitates determination with healthcare quality measures. This is accomplished by running queries on a clinical database comprising subject healthcare data. Natural language processing is utilized to extract subject healthcare data at various times from the clinical database based on individual queries, thus determining any changes in subject healthcare data over time. A rule-based component is used to implement healthcare quality measures and evaluate updated subject healthcare data based upon rules. The rules assist a healthcare provider in making deductions or choices related to subject healthcare. This is accomplished by receiving a list of data elements required for a plurality of the rules. The list of data elements includes inclusion criteria, exclusion criteria, and data required for determination to ensure compliance with the healthcare quality measures. The data elements are parsed and streamed to corresponding rules of the rule-based component based on the updated subject healthcare data. A status of subject care is obtained via an output of the rules. The status of subject care indicates whether a quality measure related to healthcare has been met for a subject and thus whether action should be taken for the subject. The healthcare quality measure is a tool that assists healthcare providers in measuring or quantifying information including healthcare processes that are associated with the ability to provide high-quality healthcare or that relate to one or more quality goals for healthcare.

It is yet another object of one or more embodiments of the present invention to provide a method for determining healthcare quality measures by evaluating subject healthcare data in real-time. The method comprises obtaining information that facilitates determination of compliance with healthcare quality measures. This is accomplished by running queries on a clinical database comprising subject healthcare data. Natural language processing is utilized to extract subject healthcare data at various times from the clinical database based on individual queries, thus determining any changes in subject healthcare data over time. A rule-based component is used to implement healthcare quality measures and evaluate updated subject healthcare data based upon rules. The rules assist a healthcare provider in making deductions or choices related to subject healthcare. This is accomplished by receiving a list of data elements required for a plurality of the rules. The list of data elements includes inclusion criteria, exclusion criteria, and data required for determination to ensure compliance with the healthcare quality measures. The data elements are parsed and streamed to corresponding rules of the rule-based component based on the updated subject healthcare data. A status of subject care is obtained via an output of the rules. The status of subject care indicates whether a quality measure related to healthcare has been met for a subject and thus whether action should be taken for the subject. The healthcare quality measure is a tool that assists healthcare providers in measuring or quantifying information including healthcare processes that are associated with the ability to provide high-quality healthcare or that relate to one or more quality goals for healthcare.

It is yet another object of one or more embodiments of the present invention to provide a system configured for determining quality measures related to healthcare by evaluating subject healthcare data in real-time. The system comprises means for obtaining information that facilitates determination to ensure compliance with healthcare quality measures. This is accomplished by running queries on a clinical database comprising subject healthcare data. Natural language processing is utilized to extract subject healthcare data at various times from the clinical database based on individual queries, thus determining any changes in subject healthcare data over time. The system further comprises means for using a rule-based component to implement healthcare quality measures and evaluate updated subject healthcare data that is updated based upon rules. The rules assist a healthcare provider in making deductions or choices related to subject healthcare. This is accomplished by receiving a list of data elements required for a plurality of the rules. The list of data elements includes inclusion criteria, exclusion criteria, and data required for determination to ensure compliance with the healthcare quality measures. The data elements are parsed and streamed to corresponding rules of the rule-based component based on the updated subject healthcare data. A status of subject care is obtained via an output of the rules. The status of subject care indicates whether a quality measure related to healthcare has been met for a subject and thus whether action should be taken for the subject. The healthcare quality measure is a tool that assists healthcare providers in measuring or quantifying information including healthcare processes that are associated with the ability to provide high-quality healthcare or that relate to one or more quality goals for healthcare.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 illustrates a system configured for determining healthcare quality measures by evaluating subject healthcare data in real-time, in accordance with one or more embodiments;

FIG. 2 illustrates a user interface showing compliance results for several subjects based on various healthcare quality measures, in accordance with one or more embodiments;

FIG. 3 is a network diagram showing a rules manager, in accordance with one or more embodiments;

FIG. 4 illustrates a method for rule-based implementation and evaluation of clinical quality, in accordance with one or more embodiments.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

As mentioned above, information on individual subject care is generally unavailable in real-time (or near real-time), thus hindering effective, guideline-based decision-making Having access to this information on individual subject care would facilitate better-informed care services for the target subject rather than future actions based on quality measures. This would result in better quality of care and improved compliance with CMS quality measures, or other quality measures.

Therefore, in this disclosure, systems and methods are described that can access subject information, determine which measures are applicable to the subject, and determine whether subject care is in compliance or not. Exemplary embodiments may aggregate the results of the subjects to determine compliance statistics for the unit and the hospital (or other medical facility). Exemplary embodiments may display the result to the staff and provide alerts for subjects whose care is not in compliance to the quality measure recommendations. Exemplary embodiments may perform these determinations automatically and display results in real-time (or near real-time) so nurses, physicians, and other caregivers may take appropriate steps to be in compliance with quality measures. The system may be set up to provide prompt warning of deviation from evidence-based guidelines. In this manner the present technology overcomes the issue of unavailability of real-time compliance statistics.

A major challenge in existing techniques for determining healthcare quality measure compliance statistics is in determining which measures are applicable to a specific subject. Usually, expert knowledge is needed to read through subject notes to judge if the subject meets the inclusion criteria for the measure. In some embodiments according to the present technology, natural language processing is utilized to identify keywords from subject notes that would indicate that the subject either meets the inclusion criteria or satisfies one of the exclusion criteria. In case information is missing, the system may alert caregivers about missing data.

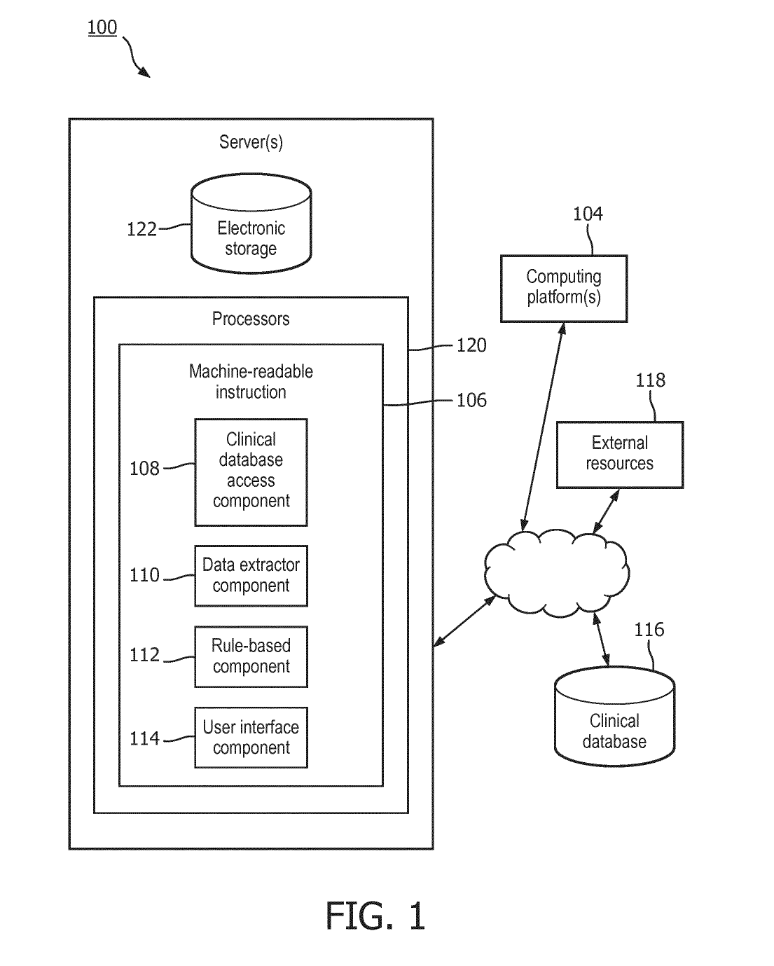

FIG. 1 illustrates an exemplary embodiment of a system 100 configured for determining healthcare quality measures by evaluating subject healthcare data in real-time, in accordance with one or more embodiments. In some embodiments, the term “real-time” may refer to “near real-time.” For purposes of brevity, “near real-time” will not always be stated. System 100 may be referred to as a quality measure implementation system, in some embodiments. System 100 is configured to provide a framework using rule-based methodologies to determine quality measures by evaluating subject parameters in real-time. Determination of clinical quality compliance is performed using a real-time rule-based methodology. Some embodiments of an exemplary quality measure implementation system are described. Arrows show the flow of data through the system. Some embodiments according to the present technology also describe methods to intuitively present the quality compliance measures to relevant stakeholders. In some embodiments, system 100 may include one or more servers. The server(s) may be configured to communicate with one or more client computing platforms according to a client/server architecture. The users may access system 100 via client computing platform(s).

In the exemplary embodiment of FIG. 1, system 100 includes one or more servers 102. The server(s) 102 may be configured to communicate with one or more computing platforms 104 according to a client/server architecture, a peer-to-peer architecture, and/or other architectures. Computing platforms 104 include, for example, a general purpose or special purpose computer system. The users may access system 100 via computing platform(s) 104.

The server(s) 102 may be configured to execute machine-readable instructions 106. The machine-readable instructions 106 may include one or more of a clinical database access component 108, a data extractor component 110, a rule-based component 112, a user interface component 114, and/or other machine-readable instruction components.

The clinical database access component 108 may be configured to provide information to and receive information from a clinical database 116. The clinical database 116 is configured for storing information, for example, subject-related data or any other type of data (medical or otherwise). The data extractor component 110 is configured to extract structured and/or unstructured data from information stored by clinical database 116 such as EHRs and/or other information. The data extractor component 110 may perform database queries of clinical database 116. In some embodiments, data extractor component 110 is configured to use a natural language processing pipeline to extract clinical concepts from notes and reports. These queries may be repeated at given time intervals (whether the same or varying in length). More specifically, in some embodiments, data extractor component 110 may obtain information that facilitates determination and ensures compliance with quality measures related to subject healthcare by running queries on clinical database 116 comprising subject healthcare data. The data extractor component 110 may utilize natural language processing to extract subject healthcare data at various times from clinical database 116 based on individual queries, thus determining any changes in subject healthcare data over time. The queries are separated by time intervals that may be the same or may differ, in some embodiments. Data extractor 110 may extract updated subject data from at least one of the queries utilizing natural language processing.

In some embodiments, data extractor component 110 extracts data that may be useful for determining compliance with healthcare quality measures while dealing with different data types. Examples of these data types may include one or more of unstructured data (e.g., free text), structured data (e.g., measured values), semi-structured data (e.g., text data such meds), and/or other types of data. System 100 receives data from clinical database 116 through queries that may be run frequently. This ensures that system 100 receives updated subject healthcare information on a frequent basis. For the various subjects, information on admission diagnosis, relevant medical history, medications, procedures, demographic information, and other data are extracted using natural language processing.

Rule-based component 112 may be configured to implement quality measures related to healthcare. Rule-based component 112 may be configured to evaluate updated subject healthcare data based upon rules. The rules assist a healthcare provider in making deductions or choices related to subject healthcare. A list of data elements, required for a plurality of the rules, is received from data extractor component 110. The list of data elements may include but are not limited to one or more of inclusion criteria, exclusion criteria, and/or data required for determination to ensure compliance with quality measures related to subject healthcare.

User interface component 114 may be configured to effectuate presentation of a user interface configured to convey to a user one or more of whether a quality measure is relevant for a given subject, whether subject data is available, whether subject care is in compliance with quality measures, and/or other information. In some embodiments, user interface component 114 may include one or more of hardware, software, firmware, and/or other items used to facilitate the workings of a user interface. In some embodiments, user interface component 114 is a user interface, action, and alert system. User interface component 114 may display compliance information for various subjects and also send alerts to caregivers to notify them of non-compliance.

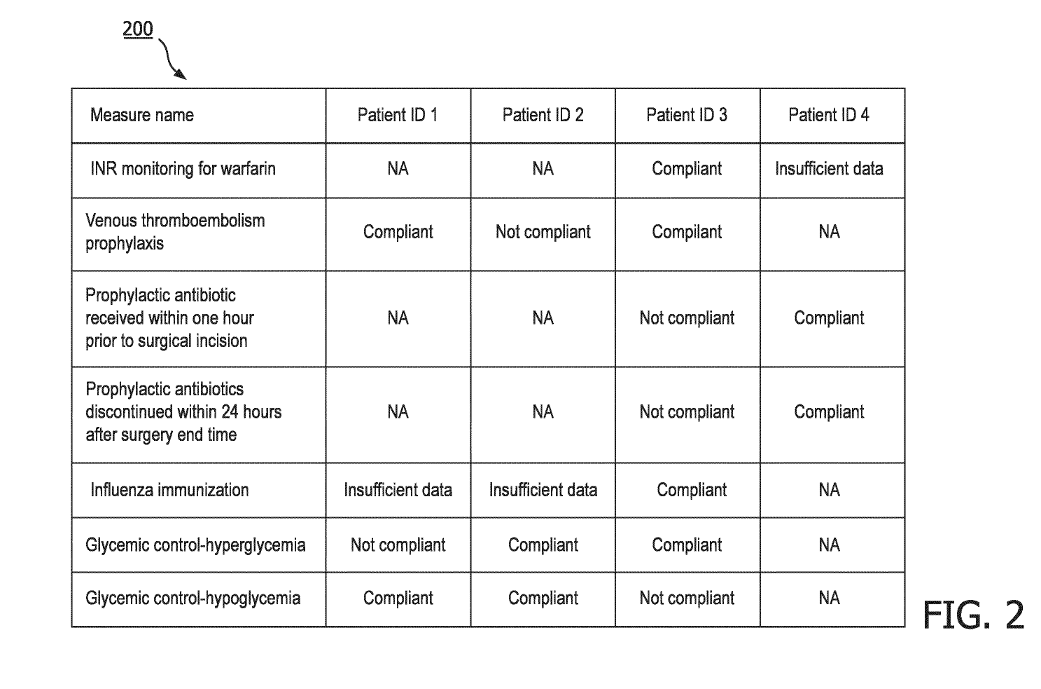

FIG. 2 illustrates user interface 200 showing compliance results for several subjects based on various quality measures, in accordance with one or more embodiments. Exemplary user interface 200 may be provided by user interface component 114. Exemplary user interface 200 shows a table of the compliance results for four subjects on seven quality measures. Users can click various cells of the table to obtain more details about a given result.

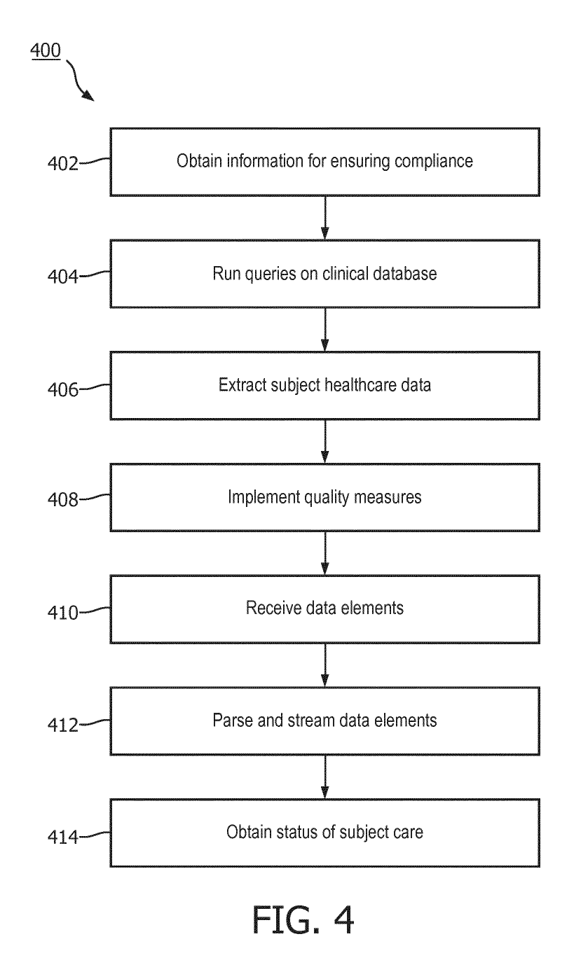

User interface component 114 has been mentioned herein. Another facet of the present technology relates to user interface component 114 for output and user interactions. The output is received by user interface component 114 from rule-based component 114, as depicted in FIG. 1. For the various measures applied to the various subjects, there are four possible outputs that may be provided. These outputs may include: subject care is in compliance with a measure; subject care is not in compliance with a measure; there is insufficient data to evaluate whether subject care is in compliance with a measure; and the measure is not applicable for the particular subject (see FIG. 4). It is envisioned that there could be fewer or more than four possible outputs in some embodiments. User interface component 114 displays the determined outputs for the various subjects in a tabular manner in some embodiments, as shown in FIG. 2, via user interface 200. However, it is contemplated that user interface component 114 may display the determined outputs for the various subjects in other ways. For example, in some embodiments the outputs may be displayed by implementing one or more of a unified user interface, integrating with EMR, and/or implementing a clinical dashboard. It should be noted that in some exemplary embodiments, if subject care is not in compliance or information is not available, a notification of such could be entered into the healthcare record of the subject.

In some embodiments, another aspect of user interface component 114 is the ability to send alerts. System 100 may alert the user when subject care might be heading toward non-compliance with one or more measures. For example if a post-surgical subject should receive antibiotics up until 24 hours after surgery and the subject is in hour 23 after surgery, system 100 may send one or more alerts to the appropriate care giver(s). This way system 100 may help improve compliance statistics. System 100 may also be programmed to send alerts when data elements are missing or subject care is not in compliance. System 100 may also allow for programming so that healthcare staff receives alerts for measures in which they are interested.

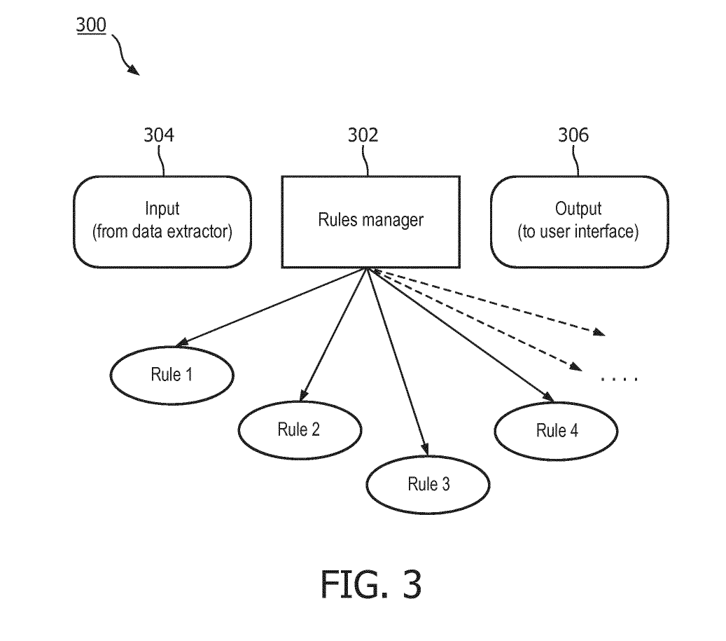

FIG. 3 illustrates a network diagram 300 showing a rules manager 302, in accordance with one or more embodiments. Rules manager 302 may be included in rule-based component 112, although it is contemplated that it may be included elsewhere. Rule-based component 112 implements the rules and evaluates the input data on these rules. The quality measures are implemented as a set of rules. These rules are managed by a rule management system such as Drools, or any other suitable system. Some other examples of companies that have their own rules engines that could be implemented in accordance with the present technology would include one or more of SAP, IBM, Oracle, and/or Microsoft. Rules manager 302 interacts with data extractor component 110 and receives input (data) 304 from data extractor component 110. Rules manager 302 also receives a list of data elements required for various rules. As mentioned herein, this list may include one or more of inclusion criteria, exclusion criteria, and/or a list of data required for determination. Rules manager 302 parses and sends the data to the appropriate rules based on the received list, as illustrated in FIG. 3. The parsed data received by the various rules is processed as described herein and shown in FIG. 4. Various rules output the status of subject care, which is sent to rules manager 302. Rules manager 302 compiles the output received from many rules run for multiple subjects and sends output (data) 306 (the results) to user interface component 114. Advantageously, many clinical quality measures may be evaluated efficiently in real-time or near real-time.

Rules manager 302 obtains, via an output of the rules, a status of subject care. The status of subject care may indicate whether a quality measure related to healthcare has been met for a subject and thus whether action should be taken for the subject. The quality measure related to healthcare may be a tool that assists healthcare providers in measuring or quantifying information. This information may include healthcare processes that are associated with the ability to provide high-quality healthcare or that relate to one or more quality goals for healthcare. The goals may include but are not limited to one or more of effective, safe, efficient, subject-centered, equitable, and/or timely care, etc.

Referring again to FIG. 1, system 100 is able to identify subjects who have a high likelihood to be non-compliant with certain protocols. For example, a quality measure may require antibiotics to be stopped after 24 hours following surgery and a post-surgical subject may be receiving antibiotics at hour 23. In this situation, system 100 may alert caregivers (e.g., healthcare providers) about impending non-compliance 30 minutes (or any other period of time) before the time window expires. This may help clinicians in better management of subject care and to provide quality care.

In this disclosure, several new methods are disclosed for determining compliance with healthcare quality measures for subjects with missing data. One strategy that may be used to decide whether a quality measure is applicable for a specific subject when information is incomplete involves extracting information on chronic conditions from past medical history. These chronic conditions are assumed to be still present, and relevant measures are determined based on this assumption. A second strategy may be used in the case that data required to assess compliance is missing. For example a measure may require glucose level measurements to be made for diabetic subjects and a diabetic subject's glucose value may be missing. In this case system 100 may search for orders for glucose measurements and use it to assess whether the quality measure is satisfied. A third strategy may be used in cases where it is difficult to determine if a quality measure is relevant for a subject with the currently available data. In this situation, a subject similarity search may be used to locate similar subjects and use that information to determine quality measure relevance.

A rules engine, such as, for example, that of rule-based component 112, may be communicatively coupled with data extractor component 110 as mentioned herein. Rule-based component 112 may implement a quality measure(s). In some embodiments, rule-based component 112 may perform rule-based implementation of the quality measure(s), including implementing rules and evaluating the updated subject data based upon the rules. This may be accomplished by receiving a list of data elements required for a plurality of the rules. The list of data elements may include one or more of lab measurements, medication administration, orders for labs and medications, diagnoses, patient history and chronic conditions, demographic information, interventions and/or other elements that may be required for quality measure evaluation. Subsequently, rules-based component 112 may perform a parsing of the data elements and send the data elements to appropriate rules based on the updated subject data. Rule-based component 112 may obtain, via an output of the rules, a status of subject care to determine a possible quality measure to be taken (implemented) for a subject.

The quality measures are formulated for evaluation on a population of subjects; therefore they should to be reformulated such that they can be applied in real-time or near real-time to a single subject. A plurality of the clinical quality measures has three elements: a numerator element, which is the number of subjects who satisfy the measure; a denominator element, which is the number of subjects for whom the measure is applicable; and an exclusion criteria conditions list. To formulate the measure such that it can be evaluated on a single subject, the second and third elements of the quality measures may be used to create a list of inclusion and exclusion criteria. The second (denominator) element specifies subjects on whom the measure is applicable. These subjects' features may be used to derive a list of inclusion criteria. The third element of the quality measures specifies conditions which, if present, mean the subject should not be included in calculating quality measure compliance. This may be used to create a list of exclusion criteria. These lists are used to evaluate the first question (measure relevance). Once the measure has been evaluated to be relevant to the subject, the next step determines whether all data required for evaluation is available. This is accomplished by creating a list of required data (information) using the first and second element of a plurality of quality measures. If the requisite data to evaluate the measure exists, system 100 determines whether subject care is in compliance. The output of this process may be displayed to a user via user interface component 114 and user interface 200.

In some cases, there might be insufficient data to determine if a quality measure is applicable or not for a specific subject. In these cases past medical history information may be used to obtain information on chronic conditions. This information may be used to determine if the subject meets any of the exclusion criteria for the measure or not. If the subject does meet the exclusion criteria for the measure, they will be included. In certain cases, some lab values that are required to calculate the measure might be missing. In these situations, system 100 may check if orders for measuring those labs are placed. In case the orders are present, system 100 may use this information to assess quality measure compliance. Another challenging issue may arise when it is difficult to decide whether a measure is applicable or not based on the given data. One example of this would be a subject whose primary diagnosis is not clear. In this situation, a subject similarity search may be applied to compare the current subject with past subjects having a similar set of labs, vitals, and other parameters. Based on a similarity score, the current subject may or may not be evaluated on the quality measure.

It is noteworthy that in some embodiments, system 100 allows for user actions and may generate alerts. For example, system 100 may allow a user to view the underlying data used to determine the output. A user may access the information that was used to determine if a given measure was relevant or not, view the missing data elements if any, and see why subject care is not in compliance. It is contemplated that system 100 may also determine a confidence score for various measures and alerts may programmed to be sent only for assessments with high confidence. In other words, a threshold confidence level may be set. This could serve to reduce the number of alerts, thus increasing efficiency in a healthcare or other facility.

The user may have the ability to mark one or more results that they think are erroneous and indicate (in the EHR) why the result(s) are wrong. This information may be either an error in extracting information from EHR (i.e., an error in data extractor component 110) or an incorrect evaluation by a rule (i.e., an error in rule-based component 112). Depending on where a particular error is, the appropriate action(s) may be taken. If the error was due to incorrect data extraction, one or more of structured query language (SQL), non-relational structured query language (NoSQL), and/or or free-text queries used to extract data may be updated. If the error was due to incorrect rule evaluation, rule-based component 106 may learn from user input to build a better model for evaluating quality measure compliance. The errors indicated in the EHR by the users are collected as negative cases and, combined with the accurate results from the present system, used to generate a training data set. The data set is then used to generate a machine-learning model (e.g., based on a decision tree or random forest algorithm) that can be integrated to augment the existing rules and/or the information extraction module.

Some embodiments according to the present technology relate to the quality measures at discharge (i.e., discharge from a hospital, medical facility, etc.). There may be multiple quality measures for discharge. These quality measures may involve prescribing certain medications, subject education, tracking subject wellbeing after discharge, etc. System 100 may be used to implement and evaluate discharge quality measures with a minor modification(s) to system 100. Data extractor component 110 may remain the same in some embodiments. The discharge quality measures may be implemented as rules in rule-based component 112. User interface component 114 may be triggered as a part of the subject discharge process. As shown in FIG. 2, user interface component 114 may visually depict which discharge measures are applicable or not applicable for a given subject, as well as which have been satisfied or not satisfied. This methodology ensures that the various quality measures are implemented and documented during the subject stay.

Another feature of the present technology, according to some embodiments, is the ability to determine unit level and hospital level compliance statistics. A unit refers to a clinical unit such as cardiac intensive care unit (ICU), general ward, or the like. Based on compliance calculations of rule-based component 106, system 100 may calculate the compliance statistics for the entire unit or hospital, or a portion(s) thereof. This data may be used to display compliance statistics of various quality measures in different locations and over different periods of time. Hence the present technology helps in both real-time and near real-time management of clinical quality measures and retrospective analysis. Retrospective analysis refers to what is currently done in hospitals, where patient data from the past year (or some portion of the year) is extracted and quality measures are evaluated. This analysis may be used to track compliance on a monthly basis or to compare performance of two units in the hospital (such as two general wards) and other forms of analyses that are currently performed.

In some embodiments according to the present technology, a detectability aspect is envisioned. System 100 may include two backend components (data extractor component 110 and rule-based component 112) and a frontend component (user interface component 114). The implementation of rule-based methodology to assess quality measure compliance using real-time data on a single subject may be detected in a competitor product. Detectability may be accomplished by investigating whether competitors use real-time or retrospective data in computing compliance with quality measures. The usage of subject similarity and the assumptions for chronic disease used to decide in cases with insufficient data can also be detected in a competitor product.

It is noteworthy that the present technology may have myriad applications. The systems and methods according to the present technology will be an invaluable tool to implement and support CMS. The present technology may provide additional value to Philips products including one of more of the eCare Manager, TASY, and/or the Phoenix dashboard of the Subject Analytics Platform. The technology can exist as an application in the Philips Collaborative Health Suite. Providing real-time or near real-time status of subject care compliance aids in actively managing quality measure compliance and quick identification of issues in workflow and communication. It is a valuable tool both for clinicians and hospital administrators, among others, and its output may be directly tied to reimbursement for a hospital etc.

In addition to implementing CMS quality measures, system 100 provides a robust framework to apply quality measures developed by other organizations such as the Joint Commission, National Institute for Heath and Care Excellence (NICE) in UK or measures that are implemented by the hospitals themselves. Therefore, this invention may advantageously be used as a healthcare quality control tool.

In some embodiments, server(s) 102, computing platform(s) 104, clinical database 116, and/or external resources 118 may be operatively linked via one or more electronic communication links. For example, such electronic communication links may be established, at least in part, via a network such as the Internet and/or other networks. It will be appreciated that this is not intended to be limiting, and that the scope of this disclosure includes embodiments in which server(s) 102, computing platform(s) 104, and/or external resources 118 may be operatively linked via some other communication media.

A given computing platform 104 may include one or more processors configured to execute machine-readable instructions. The machine-readable instructions may be configured to enable an expert or user associated with the given computing platform 104 to interface with system 100 and/or external resources 118, and/or provide other functionality attributed herein to computing platform(s) 104. By way of non-limiting example, a given computing platform 104 may include one or more of a desktop computer, a laptop computer, a handheld computer, a tablet computing platform, a netbook, a smartphone, a gaming console, and/or other computing platforms.

External resources 118 may include sources of information, hosts and/or providers of electronic health records (EHRs), external entities participating with system 100, and/or other resources. In some embodiments, some or all of the functionality attributed herein to external resources 118 may be provided by resources included in system 100.

Server(s) 102 may include electronic storage 122, one or more processors 120, and/or other components. Server(s) 102 may include communication lines, or ports to enable the exchange of information with a network and/or other computing platforms. Illustration of server(s) 102 in FIG. 1 is not intended to be limiting. Server(s) 102 may include a plurality of hardware, software, and/or firmware components operating together to provide the functionality attributed herein to server(s) 102. For example, server(s) 102 may be implemented by a cloud of computing platforms operating together as server(s) 102.

Electronic storage 122 may comprise non-transitory storage media that electronically stores information. The electronic storage media of electronic storage 122 may include one or both of system storage that is provided integrally (i.e., substantially non-removable) with server(s) 102 and/or removable storage that is removably connectable to server(s) 102 via, for example, a port (e.g., a USB port, a firewire port, etc.) or a drive (e.g., a disk drive, etc.). Electronic storage 122 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 122 may include one or more virtual storage resources (e.g., cloud storage, a virtual private network, and/or other virtual storage resources). Electronic storage 122 may store software algorithms, information determined by processor(s) 120, information received from server(s) 102, information received from computing platform(s) 104, and/or other information that enables server(s) 102 to function as described herein.

Processor(s) 120 may be configured to provide information processing capabilities in server(s) 102. As such, processor(s) 120 may include one or more of a digital processor, an analog processor, a digital circuit designed to process information, an analog circuit designed to process information, a state machine, and/or other mechanisms for electronically processing information. Although processor(s) 120 is shown in FIG. 1 as a single entity, this is for illustrative purposes only. In some embodiments, processor(s) 120 may include a plurality of processing units. These processing units may be physically located within the same device, or processor(s) 120 may represent processing functionality of a plurality of devices operating in coordination. The processor(s) 120 may be configured to execute one ore more of machine-readable instruction components 108, 110, 112, 114, and/or other machine-readable instruction components. Processor(s) 120 may be configured to execute one or more of machine-readable instruction components 108, 110, 112, 114, and/or other machine-readable instruction components by software; hardware; firmware; some combination of software, hardware, and/or firmware; and/or other mechanisms for configuring processing capabilities on processor(s) 120. As used herein, the term “machine-readable instruction component” may refer to any component or set of components that perform the functionality attributed to the machine-readable instruction component. This may include one or more physical processors during execution of processor readable instructions, the processor readable instructions, circuitry, hardware, storage media, or any other components.

It should be appreciated that although machine-readable instruction components 108, 110, 112, and 114 are illustrated in FIG. 1 as being implemented within a single processing unit, in embodiments in which processor(s) 120 includes multiple processing units, one or more of machine-readable instruction components 108, 110, 112, and/or 114 may be implemented remotely from the other machine-readable instruction components. The description of the functionality provided by one or more of machine-readable instruction components 108, 110, 112, and/or 114 described below is for illustrative purposes, and is not intended to be limiting, as any of one or more of machine-readable instruction components 108, 110, 112, and/or 114 may provide more or less functionality than is described. For example, one or more of machine-readable instruction components 108, 110, 112, and/or 114 may be eliminated, and some or all of its functionality may be provided by other ones of one or more of machine-readable instruction components 108, 110, 112, and/or 114. As another example, processor(s) 120 may be configured to execute one or more additional machine-readable instruction components that may perform some or all of the functionality attributed below to one or more of machine-readable instruction components 108, 108, 110, 112, and/or 114.

FIG. 4 illustrates a method 400 for rule-based embodiment and evaluation of clinical quality, in accordance with one or more embodiments. The operations of method 400 presented below are intended to be illustrative. In some embodiments, method 400 may be accomplished with one or more additional operations not described, and/or without one or more of the operations discussed. Additionally, the order in which the operations of method 400 are illustrated in FIG. 4 and described below is not intended to be limiting.

In some embodiments, one or more operations of method 400 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 400 in response to instructions stored electronically on an electronic storage medium. The one or more processing devices may include one or more devices configured through hardware, firmware, and/or software to be specifically designed for execution of one or more of the operations of method 400.

At an operation 402, obtain information that facilitates determination to ensure compliance with quality measures related to subject healthcare. In some embodiments, obtaining the information may include one or both of operations 404 and 406. Operation 402 may be performed by one or more hardware processors 120 configured to execute a machine-readable instruction component that is the same as or similar to one or more of components 108, 110, 112, and/or 114 (as described in connection with FIG. 1), in accordance with one or more implementations.

At operation 404, queries are run on clinical database 116 comprising subject healthcare data. Operation 404 may be performed by one or more hardware processors 120 configured to execute a machine-readable instruction component that is the same as or similar to one or more of components 108, 110, 112, and/or 114 (as described in connection with FIG. 1), in accordance with one or more implementations.

At operation 406, natural language processing is utilized to extract subject healthcare data at various times from the clinical database based on individual queries, thus determining any changes in subject healthcare data over time. Operation 406 may be performed by one or more hardware processors 120 configured to execute a machine-readable instruction component that is the same as or similar to one or more of components 108, 110, 112, and/or 114 (as described in connection with FIG. 1), in accordance with one or more implementations.

At an operation 408, quality measures related to healthcare may be implemented such that updated subject healthcare data may be evaluated based upon rules. In some embodiments, this implementation of quality measures may include one or more of operations 410, 412, and/or 416. The rules may assist a healthcare provider in making deductions or choices related to subject healthcare. Operation 408 may be performed by one or more hardware processors 120 configured to execute a machine-readable instruction component that is the same as or similar to one or more of components 108, 110, 112, and/or 114 (as described in connection with FIG. 1), in accordance with one or more implementations.

At operation 410, a list of data elements required for a plurality of the rules may be received, the list of data elements including one or more of inclusion criteria, exclusion criteria, and/or data required for determination to ensure compliance with quality measures related to subject healthcare. Operation 410 may be performed by one or more hardware processors 120 configured to execute a machine-readable instruction component that is the same as or similar to one or more of components 108, 110, 112, and/or 114 (as described in connection with FIG. 1), in accordance with one or more implementations.

At operation 412, the data elements may be parsed and streamed to corresponding rules of rule-based component 112 based on the updated subject data. Operation 412 may be performed by one or more hardware processors 120 configured to execute a machine-readable instruction component that is the same as or similar to one or more of components 108, 110, 112, and/or 114 (as described in connection with FIG. 1), in accordance with one or more implementations.

At operation 414, via an output of the rules, a status of subject care may be obtained via an output of the rules; the status of subject care indicating whether a quality measure related to healthcare has been met for a subject and thus whether action should be taken for the subject. The quality measure related to healthcare may be a tool that assists healthcare providers in measuring or quantifying information including healthcare processes that are associated with the ability to provide high-quality healthcare or that relate to one or more quality goals for healthcare. The goals may include one or more of effective, safe, efficient, subject-centered, equitable, and/or timely care. Operation 414 may be performed by one or more hardware processors configured to execute a machine-readable instruction component that is the same as or similar to one or more of components 108, 110, 112, and/or 114 (as described in connection with FIG. 1), in accordance with one or more implementations.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A system configured for determining healthcare quality measures by evaluating subject healthcare data in real-time, the system comprising:
one or more hardware processors configured by machine-readable instructions to:
obtain information that facilitates determination of compliance with healthcare quality measures by:
running queries on a clinical database comprising subject healthcare data; and
utilizing natural language processing to extract subject healthcare data at various times from the clinical database based on individual queries, thus determining any changes in subject healthcare data over time; and
use a rule-based component to implement healthcare quality measures and evaluate subject healthcare data that is updated based upon rules, the rules assisting a healthcare provider in making deductions or choices related to subject healthcare, by:
receiving a list of data elements required for a plurality of the rules, the list of data elements including: inclusion criteria, exclusion criteria, and data required for determination of compliance with the healthcare quality measures;
parsing and streaming the data elements to corresponding rules of the rule-based component based on the updated subject healthcare data; and
obtaining, based on the rules, a status of subject care, the status of subject care indicating whether the healthcare quality measure has been met for a subject and thus whether action should be taken for the subject, the healthcare quality measure being a tool that assists healthcare providers in measuring or quantifying information including healthcare processes that are associated with the ability to provide high-quality healthcare or that relate to one or more quality goals for healthcare.

2. The system of claim 1, wherein the one or more hardware processors are further configured to evaluate, via the use of the rules, whether a given quality measure is relevant for a given subject.

3. The system of claim 1, wherein the one or more hardware processors are further configured to cause the rules to, if a given quality measure is relevant for the given subject, evaluate whether subject data is available to assess subject care compliance with the given quality measure.

4. The system of claim 1, wherein the one or more hardware processors are further configured to evaluate, via the use of one or more rules, if the subject data is available, and evaluate whether the quality measure is satisfied for the given subject.

5. The system of claim 1, wherein the one or more hardware processors are further configured to effectuate presentation of a user interface configured to convey to a user whether a quality measure is relevant, whether subject data is available, and whether subject care is in compliance with quality measures.

6. A method for determining healthcare quality measures by evaluating subject healthcare data in real-time, the method comprising:
obtaining information that facilitates determination of compliance with healthcare quality measures related to subject healthcare by:
running queries on a clinical database comprising subject healthcare data; and
utilizing natural language processing to extract subject healthcare data at various times from the clinical database based on individual queries, thus determining any changes in subject healthcare data over time;
using a rule-based component to implement healthcare quality measures and evaluate updated subject healthcare data based upon rules, the rules assisting a healthcare provider in making deductions or choices related to subject healthcare, by:
receiving a list of data elements required for a plurality of the rules, the list of data elements including inclusion criteria, exclusion criteria, and data required for determination of compliance with the healthcare quality measures;
parsing and streaming the data elements to corresponding rules of the rule-based component based on the updated subject healthcare data; and
obtaining, based on the rules, a status of subject care, the status of subject care indicating whether a the healthcare quality measure has been met for a subject and thus whether action should be taken for the subject, the healthcare quality measure being a tool that assists healthcare providers in measuring or quantifying information including healthcare processes that are associated with the ability to provide high-quality healthcare or that relate to one or more quality goals for healthcare.

7. The method of claim 6, further comprising evaluating, via the use of the rules, whether a given quality measure is relevant for a given subject.

8. The method of claim 6, further comprising causing the rules to, if a given quality measure is relevant for the given subject, evaluate whether subject data is available to assess subject care compliance with the given quality measure.

9. The method of claim 6, further comprising evaluating, via the use of one or more rules, if the subject data is available, and evaluating whether the quality measure is satisfied for the given subject.

10. The method of claim 6, wherein the one or more hardware processors are further configured to effectuate presentation of a user interface configured to convey to a user whether a quality measure is relevant, whether subject data is available, and whether subject care is in compliance with quality measures.

11. A system configured for determining healthcare quality measures by evaluating subject healthcare data in real-time, the system comprising:
means for obtaining information that facilitates determination of compliance with healthcare quality measures related to subject healthcare by:
running queries on a clinical database comprising subject healthcare data; and
utilizing natural language processing to extract subject healthcare data at various times from the clinical database based on individual queries, thus determining any changes in subject healthcare data over time;
means for using a rule-based component to implement healthcare quality measures and evaluate updated subject healthcare data that is updated based upon rules, the rules assisting a healthcare provider in making deductions or choices related to subject healthcare, by:
receiving a list of data elements required for a plurality of the rules, the list of data elements including inclusion criteria, exclusion criteria, and data required for determination of compliance with the healthcare quality measures;
parsing and streaming the data elements to corresponding rules of the rule-based component based on the updated subject healthcare data; and
obtaining, based on the rules, a status of subject care, the status of subject care indicating whether the healthcare quality measure has been met for a subject and thus whether action should be taken for the subject, the healthcare quality measure related to healthcare being a tool that assists healthcare providers in measuring or quantifying information including healthcare processes that are associated with the ability to provide high-quality healthcare or that relate to one or more quality goals for healthcare.

12. The system of claim 11, further comprising means for evaluating, via the use of the rules, whether a given quality measure is relevant for a given subject.

13. The system of claim 11, further comprising means for causing the rules to, if a given quality measure is relevant for the given subject, evaluate whether subject data is available to assess subject care compliance with the given quality measure.

14. The system of claim 11, further comprising means for evaluating, via the use of one or more rules, if the subject data is available, and evaluate whether the quality measure is satisfied for the given subject.

15. The system of claim 11, further comprising means for effectuating presentation of a user interface configured to convey to a user whether a quality measure is relevant, whether subject data is available, and whether subject care is in compliance with quality measures.
